(19) United States
(12) Patent Application Publication
(10) Pub. No.: US 2009/0182422 A1
(43) Pub. Date: Jul. 16, 2009

(54) INTRAOCULAR THIN LENS FOR ANTERIOR CHAMBER INSTALLATION

(76) Inventors: Lee T. Nordan, Rancho Santa Fe, CA (US); G. Michael Morris, Victor, NY (US)

Correspondence Address: MORRISON & FOERSTER LLP, 12531 HIGH BLUFF DRIVE, SUITE 100, SAN DIEGO, CA, 92130-2040, US

(21) Appl. No.: 11/965,690

(22) Filed: Dec. 27, 2007

Related U.S. Application Data
(63) Continuation of application No. 10/443,519, filed on May 23, 2003, now abandoned.
Application No. 10/443,519 is a continuation-in-part of application No. 10/334,867, filed on Dec. 30, 2002, now abandoned.
Application No. 10/334,867 is a continuation-in-part of application No. PCT/US00/32148, filed on Nov. 27, 2000.
Application No. PCT/US00/32148 is a continuation-in-part of application No. 09/215,574, filed on Dec. 16, 1998, now Pat. No. 6,152,958.

(51) Int. Cl. A61F 2/16

(52) U.S. Cl. 623/6.25

(57) ABSTRACT

A thin foldable intraocular implant specifically configured for installation into the anterior chamber of a phakic or pseudophakic eye has broad positioning flaps that do not apply any substantial pressure against the wall of the eye. It can be rolled for insertion through a corneal incision as small as 2.75 millimeters. The implant is constituted by a two-layered resiliently flexible membrane having a corrective layer of about 50 to 130 microns and an overall thickness of about 150 to 530 microns, that vaults the iris without contacting it. The optic is constituted by a multi-order diffractive (MOD) structure, and is made of silicone, PMMA, hydrogel or hydrophobic acrylate.

### CROSS-REFERENCE TO RELATED APPLICATIONS

This is a continuation of co-pending U.S. application Ser. No. 10/443,519, filed May 23, 2003, which is a continuation-in-part of co-pending U.S. application Ser. No. 10/334,867, filed Dec. 30, 2002, which is a continuation-in-part of PCT Application No. PCT/US00/32148, filed Nov. 27, 2000, which is a continuation-in-part of U.S. application Ser. No. 09/215,574, filed Dec. 16, 1998, now U.S. Pat. No. 6,152,958.

### FIELD OF THE INVENTION

The present invention relates to intraocular implants, and more specifically to implants intended to be inserted in the anterior chamber of the eye in order to correct optical deficiencies without removal and replacement of the crystalline lens. Background Art

### BACKGROUND OF THE INVENTION

Intraocular lenses (IOLs) are routinely used nowadays for restoring vision after removal of the cataracted lens. An AOL may also be installed in the anterior chamber independently of any removal and replacement of the crystalline lens. Whether the IOL is installed in the posterior chamber of the eye in lieu of the removed cataracted lens, or in the anterior chamber, it must be small enough to pass through a minimal corneal incision. The reduction in the overall dimension of the IOL is limited, however, by the necessity of avoiding glare by providing a substitute optic that is large enough to cover the pupil when it is fully dilated for proper night time vision. One approach to reducing glare while at the same time reducing the size of the incision in the cornea is to construct the IOL from several pieces which are joined together after the individual pieces are inserted through the corneal incision as disclosed in U.S. Pat. No. 5,769,889 Kelman. The complexity of this type of IOLs, the difficulty of their post insertion assembly coupled with the required thickness and rigidity of the optic element, still force the ophthalmic surgeon into tolerable compromises between reduced size and peripheral glare coupled with impaired night vision.

Due to the fact that prior art IOLs specially those installed in the anterior chamber must be precisely tailored to the size of the eye, the surgeon must have at his disposal, a variety of size-graded IOLs, and select the one offering the closest match.

Conventional lenticular elements, whatever their size, are still subject to various spherical and thickness aberrations which are not easily correctable during the manufacture of the IOL.

The invention results from a search for a simple, preferably one-piece IOL with an optic having a diameter sufficient to cover the size of a dilated pupil, but yet insertable to a relatively small corneal incision into a wide range of eye sizes.

### SUMMARY OF THE INVENTION

The principal and secondary objects of this invention are to provide the ophthalmic surgeon with a simple, one-piece IOL which avoids the major drawbacks of the device of the prior art, particularly reduced coma, glare, impaired night vision, and blurring due to spherical and thickness aberrations, and which can be collapsed to a relatively small size for insertion through a corneal incision of about 2.75 millimeters, and which automatically adjust to the size of the eye.

These and other valuable objects are achieved by forming a thin lens inherently immune to spherical and thickness aberrations on a resiliently flexible membrane that can be rolled or folded to pass through a small corneal incision. The thin lens optic typically uses a plurality of optic rings concentric with the central zone and extends up to a total diameter of approximately 6 millimeters. The lenticular zone forms a diffractive phase Fresnel-type lens formed of concentric zones having profiles that provide a phase jump delay at each zone boundary which is a multiple of waves at the design wavelength in order to focus a plurality of different wavelengths to a single point. The membrane and its incorporated thin lens optic is arcuately shaped for adjustable installation in the anterior chamber where it vaults the iris and is stabilized by sets of flaps that nest into the corner of the chamber. Contrary to the compressed haptics commonly used to secure prior art, IOLs, the aforesaid flaps do not exert any substantial pressure upon the wall of the eye. The vaulted shape of the device combined with its thinness keep it away from the endothelium. Its neutral buoyancy prevents any pressure on the iris eliminating risks of closure, cataract or iris pigment dispersion. The large footprint of the flaps prevent synechiae and their encapsulation by the iris. The natural buoyancy of the device is improved by a plurality of fenestrations. The thin lens can be configured in a variety of successive dioptic powers, over a range from −15 to +15 diopters, in order to correct practically all types of refractive errors.

### BRIEF DESCRIPTION OF THE DRAWING

FIG. 1 is a cross-sectional view of a mamalian eye in which is implanted a corrective device according to the invention;

FIG. 2 is a front elevational view of the device;

FIG. 3 is a side elevational view thereof;

FIG. 4 is a cross-sectional view of a thin lens optic region;

FIG. 5 is a diagram of the MOD lens; and

FIG. 6 is a diffraction plot thereof.

### DESCRIPTION OF THE PREFERRED EMBODIMENT OF THE INVENTION

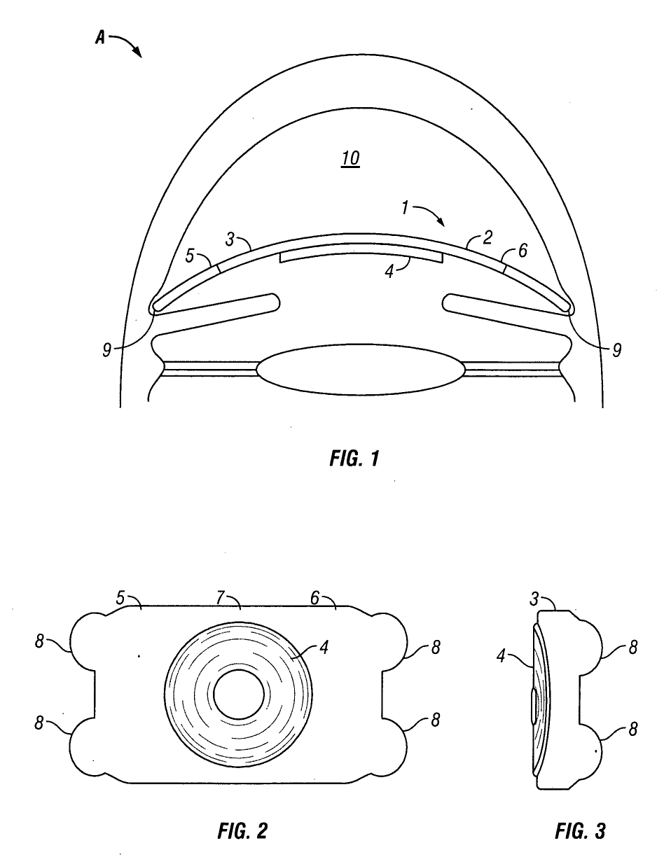

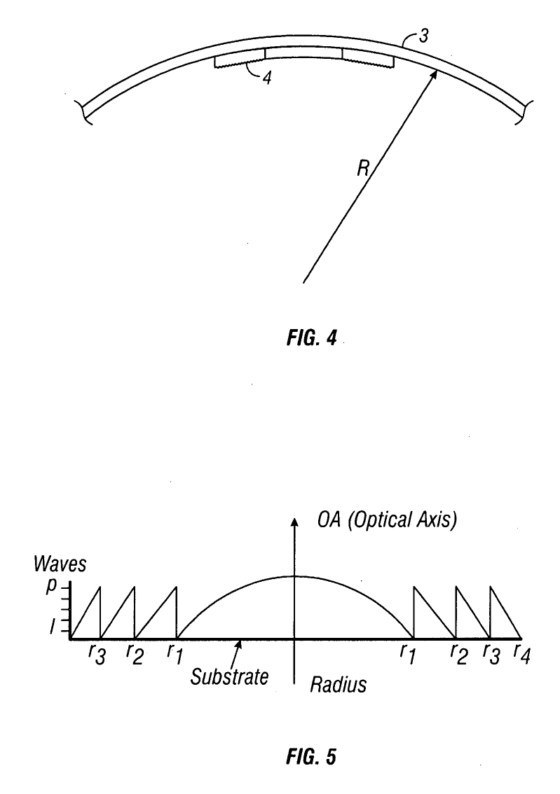

Referring now to the drawing, there is shown a surgically corrected mamalian eye A having a corrective device 1 mounted in the anterior chamber 10. The device consists essentially of a membrane 2 preferably made of flexible silicone such as Material Number MED-6820 commercially available from NuSil Silicone Technology of Carpenteria, Calif. Other resilient materials such as PMMA, hydrophobic acrylate or hydrogel could also be used. The membrane 2 is constituted by a first substrate layer 3 having no corrective properties and a second corrective layer 4 intimately pressed or bonded against the first layer and carrying the optic. The substrate layer 3 is preferably made in a thickness of approximately 100 to 400 microns. Although flexible, it returns to a rest position with a single radius curvature in a range of approximately 10 to 16 millimeters about a vertical axis. The corrective or optic layer 4 has a thickness between approximately 50 to 130 microns. The combined layers exhibit a total thickness of about 150 to 530 microns. The overall dimensions are approximately 12 millimeters in length, 8 millimeters in width. The membrane can be bent and even rolled or folded for insertion into the interior chamber through a small incision of no more than 2.75 millimeters in length. The membrane has enough resiliency to return to its prerolled or prefolded arcuate shape. The optic region 4 in the center of the membrane has an overall diameter of approximately 6 millimeters. The optic region is essentially constituted by what is called a “thin lens” in the fields of optics and ophthalmology. An example of thin lens is disclosed in U.S. Pat. No. 6,152,958 Nordan which patent is incorporated in this Specification by this references. The two lateral portions 5, 6 of the membrane astride the median optic region 7 that includes the optic layer 4 are shaped to define at least two flaps 8 designed to nest intimately into the corner 9 of the anterior chamber as illustrated in FIG. 1. Accordingly, the median portion and the lateral portion with their bent flaps 8 form a vault that spans the anterior chamber 10 in a direction substantially parallel to the iris 11. The curvature of the membrane is permanently imparted to the substrate layer 3 during the fabrication of the device.

The membrane retains an arcuate shape in its resting position. The device fits within a circle having a radius R of approximately 6.5 millimeters. Due to the flexibility of the membrane, this size can accommodate practically all eye sizes. In other words, depending upon the span of the anterior chamber 10, the device upon installation can adjust its length by decreasing or increasing its radius R of curvature within a range of about 5 to 15 millimeters.

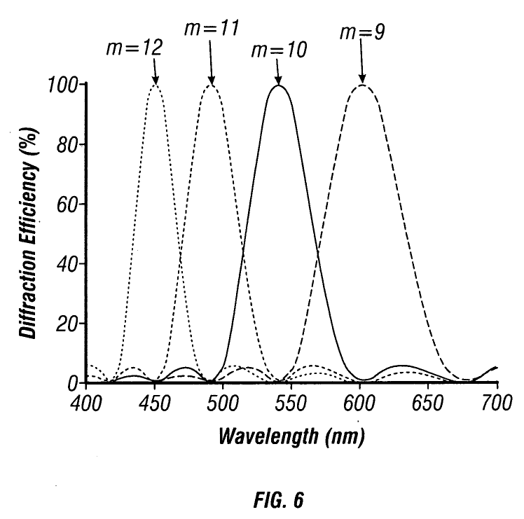

Referring now to FIGS. 5 and 6, in the preferred embodiment of the thin lens implant of the invention, the optic is constituted by a multi-order diffractive (MOD) lens having a surface geometry of the type disclosed in U.S. Pat. No. 5,589,982 Faklis et al.; which patent is hereby incorporated by reference into this specification.

As disclosed in said patent, a MOD lens is capable of focusing a plurality of different wavelengths of light to a single focus. A diffractive structure is used having a plurality of annular zones which define zone boundaries which diffract light of each of the wavelengths in a different diffractive order to the focus thereby providing a plural or multiple order diffractive singlet.

The imaging properties of a plural or multi-order diffractive (MOD) lens enable the use of the lens in conjunction with light that has either a broad spectral range or a spectrum consisting of multiple spectral bands. The MOD lens differs from standard diffractive lenses in that the phase delay or jump at the zone boundaries is a multiple of waves at the design wavelength (a multiple of 2π, i.e., φ(rⱼ)=2πp, where p is an integer greater or equal to 2, and the zone radii are obtained by solving the equation φ(rⱼ)=2πpj, where φ(r) represents the phase function for the wavefront emerging from the lens. The number of 2π phase jumps, p, represents a degree of freedom allowing an optical designer to use distinct diffraction orders to focus two or more spectral components upon the same spatial location.

Referring to FIG. 5, the blaze profile is on one side of a substrate of optically transmissive material. The number of waves for each zone boundary is indicated as p and the phase jump of phases at each zone boundary, which are at radii r1, r2, r3 and r4 is constant. The center of the lens is along the optical axis and is perpendicular to the plane of the substrate on which the profile is formed. The profile of the lens may also be a phase reversal (or Wood) profile, or a multi-level approximation to the blaze profile.

The profile may be defined between substrates, rather than on a planar surface of a substrate, as shown, where the substrates on opposite sides of the profile have different indicies of refraction. In the preferred embodiment of the invention, the profile is defined on a curved substrate.

The zone spacing or width of the zones between the zone boundaries r₁-r₂, r₂-r₃, r₃-r₄ are full period Fresnel zones.

FIG. 6 illustrates the wavelength dependence of the diffraction efficiency for a range of diffracted orders neglecting material dispersion. The peaks in diffraction efficiency occur at precisely those wavelengths nm that come to a common focus.

### EXAMPLE

A thin, foldable, MOD diffractive, polychromatic, intra-ocular implant according to the invention with the following parameters exhibits the following characteristics:
Material: NuSil-MED-6820
Thickness: optic layer 45 microns
Thickness of Substitute: 300 microns
Diameter of corrective portion: 0.65 centimeter
Power: −6 diopters
Number of concentric zones: 50

Radial location and width of each zone:

| Zone | | Radial Location | Width |
|---|---|---|---|
| Number | Phase | (millimeters) | (microns) |
| | 0 | 0.0000 | 0.000 |
| 1 | 1 | 0.42564 | 525.636 |
| 2 | 2 | 0.60194 | 176.305 |
| 3 | 3 | 0.73722 | 135.283 |
| 4 | 4 | 0.85127 | 114.049 |
| 5 | 5 | 0.95176 | 100.479 |
| 6 | 6 | 1.04259 | 90.840 |
| 7 | 7 | 1.12613 | 83.537 |
| 8 | 8 | 1.20388 | 77.753 |
| 9 | 9 | 1.27691 | 73.028 |
| 10 | 10 | 1.34598 | 69.071 |
| 11 | 11 | 1.41168 | 65.696 |
| 12 | 12 | 1.47445 | 62.772 |
| 13 | 13 | 1.53466 | 60.206 |
| 14 | 14 | 1.59259 | 57.931 |
| 15 | 15 | 1.64848 | 55.898 |
| 16 | 16 | 1.70255 | 54.063 |
| 17 | 17 | 1.75495 | 52.398 |
| 18 | 18 | 1.80582 | 50.878 |
| 19 | 19 | 1.85531 | 49.484 |
| 20 | 20 | 1.90351 | 48.198 |
| 21 | 21 | 1.95051 | 47.007 |
| 22 | 22 | 1.99641 | 45.901 |
| 23 | 23 | 2.04128 | 44.869 |
| 24 | 24 | 2.08519 | 43.903 |
| 25 | 25 | 2.12818 | 42.998 |
| 26 | 26 | 2.17033 | 42.147 |
| 27 | 27 | 2.21167 | 41.343 |
| 28 | 28 | 2.25226 | 40.585 |
| 29 | 29 | 2.29212 | 39.866 |
| 30 | 30 | 2.33131 | 39.184 |
| 31 | 31 | 2.36985 | 38.537 |
| 32 | 32 | 2.40777 | 37.920 |
| 33 | 33 | 2.44510 | 37.332 |
| 34 | 34 | 2.48187 | 36.770 |
| 35 | 35 | 2.51810 | 36.234 |
| 36 | 36 | 2.55382 | 35.719 |
| 37 | 37 | 2.58905 | 35.227 |
| 38 | 38 | 2.62380 | 34.754 |
| 39 | 39 | 2.65810 | 34.299 |
| 40 | 40 | 2.69196 | 33.863 |
| 41 | 41 | 2.72541 | 33.442 |
| 42 | 42 | 2.75844 | 33.036 |
| 43 | 43 | 2.79109 | 32.646 |
| 44 | 44 | 2.82336 | 32.267 |
| 45 | 45 | 2.85526 | 31.904 |
| 46 | 46 | 2.88681 | 31.551 |
| 47 | 47 | 2.91802 | 31.209 |
| 48 | 48 | 2.94890 | 30.880 |
| 49 | 49 | 2.97946 | 30.559 |
| 50 | 50 | 3.00791 | 30.249 |

While the preferred embodiment of the invention has been described, modifications can be made and other embodiments may be devised without departing from the spirit of the invention and the scope of the appended claims.

1. A single piece corrective device for installation in the anterior chamber of a phakic or pseudophakic eye which comprises:
a single thin, resiliently bendable membrane shaped and dimensioned to arcuately span the anterior chamber substantially parallelly to the iris;
said membrane including two layers;
a first one of said layers including a median portion, and at least two lateral portions astride said median portion, a second one of said layers including a corrective portion bonded to said median portion wherein said corrective portion includes a multi-order, diffractive thin lens; and
said thin lens comprises a discontinuous optic zone having a plurality of concentric optic rings, wherein each of said plurality of concentric optic rings has a ring boundary with an adjacent ring and said rings are shaped and dimensioned to provide a phase jump at each ring boundary for at least one spectral component of a light beam incident upon said lens, and wherein a plurality of said spectral components have a given wavelength and said rings are shaped and dimensioned to provide a phase jump equal to 2πp wherein p is an integer greater or equal to 1, said p and the widths of the rings are selected to direct said spectral components to a single focus point.

2. The device of claim 1, wherein said second layer has a thickness of approximately 50 to 130 microns.

3. The device of claim 1, wherein said first layer comprises at least two anchoring flaps each shaped and dimensioned to intimately nest into a corner of the anterior chamber.

4. The device of claim 1, wherein said median portion and lateral portions form a vault having a radius of approximately 5 to 15 millimeters.

5. The device of claim 1, wherein said layers are made of flexible silicone.

6. The device of claim 1, wherein said thin lens has correction powers in a range of approximately minus 15 diopters to plus 15 diopters.

7. The corrective device of claim 1, wherein said rings are radially spaced at radii, rⱼ obtained by solving the equation φ(rⱼ)=2πp where φ(r) represents the phase function of a wavefront emerging from said optic rings.

8. The device of claim 4, having sufficient flexibility to adjustably change said radius to match the span of said anterior chamber.

9. A method to correct visual acuity in a mammalian eye, comprising:
introducing the single piece corrective device of claim 1 into an anterior chamber of the mammalian eye, such that the visual acuity of the mammalian eye is corrected.

10. The method of claim 9, wherein said rings are radially spaced at radii, rⱼ obtained by solving the equation φ(rⱼ)=2πp where φ(r) represents the phase function of a wavefront emerging from said optic rings.

11. The method of claim 9, wherein said second layer has a thickness of approximately 50 to 130 microns.

12. The method of claim 9, wherein said layers are made of silicone.

13. The method of claim 9, wherein said layers are made of resiliently flexible materials taken from a group consisting of silicone, PMMA, hydrogel and hydrophobic acrylate.
